(19) United States
(12) Patent Application Publication
(10) Pub. No.: US 2003/0134862 A1
(43) Pub. Date: Jul. 17, 2003

(54) Compounds

(76) Inventors: Katarina Beierlein, Uppsala (SE); Anna-Lena Gustavsson, Stockholm (SE); Lena Jendeberg, Solna (SE); Bengt Lindqvist, Uppsala (SE)

Correspondence Address: JEFFREY D. HSI, Fish & Richardson P.C., 225 Franklin Street, Boston, MA, 02110-2804, US

(21) Appl. No.: 10/324,772

(22) Filed: Dec. 19, 2002

Related U.S. Application Data
(60) Provisional application No. 60/351,814, filed on Jan. 25, 2002.

(30) Foreign Application Priority Data
Dec. 20, 2001 (SE) 0104366-0

(51) Int. Cl. A61K 31/519; C07D 487/02

(52) U.S. Cl. 514/259.3; 544/281

(57) ABSTRACT

The present invention relates to novel compounds which are pyrazolo[1,5-a]pyrimidines, and which modulate the activity of peroxisome proliferator-activated receptors (PPAR) α and/or γ. The said compounds are predicted to be useful in the treatment of metabolic diseases, e.g. type II diabetes.

### CROSS REFERENCE TO RELATED APPLICATIONS

Pursuant to 35 USC §19(e), this application claims the benefit of prior U.S. provisional application No. 60/351,814, filed Jan. 25, 2002.

Pursuant to 35 USC §119, this application claims the benefit of Swedish Patent Application No. 0104366-0 filed Dec. 20, 2001.

### TECHNICAL FIELD

The present invention relates to novel compounds which are pyrazolo[1,5-a]pyrimidines, and which modulate the activity of peroxisome proliferator-activated receptors (PPAR) α and/or γ. The said compounds are predicted to be useful in the treatment of metabolic diseases, e.g. type II diabetes.

### BACKGROUND

In developed societies, chronic diseases such as diabetes, obesity, atherosclerosis and cancer are responsible for most deaths. These ailments have complex causes involving genetic, environmental and nutritional factors. There is evidence that a group of closely related nuclear receptors, called peroxisome proliferator-activated receptors (PPARs), may be involved in these diseases. This, together with the fact that drugs such as thiazolidinediones and fibrates can modulate PPAR activity, has instigated a huge research effort into PPARs. For reviews on PPARs and their medical significance, see e.g. Kersten, S. et al. (2000) Nature 405:421-424; Willson, T. M. et al. (2000) J. Med. Chem. 43:527-550; Vamecq, J. et al. (1999) Lancet 354:141-148.

The PPARs were first cloned as the nuclear receptors that mediate the effects of synthetic compounds called peroxisome proliferators on gene transcription. It soon became clear that eicosanoids and fatty acids could also regulate gene transcription through PPARs. At the molecular level, PPARs act in a similar manner to other nuclear hormone receptors. First, they bind a specific element in the promoter region of target genes. PPAR and some other nuclear hormone receptors bind the promoter only as a heterodimer with the receptor for 9-cis retinoic acid, RXR (retinoid X receptor). Second, they activate transcription in response to binding of the hormone (ligand). For the PPAR:RXR heterodimer, binding of the ligand of either receptor can activate the complex, but binding of both ligands simultaneously is more potent.

Three PPAR isotypes have been identified: α, β (also called δ and NUCI) and γ. PPARα (GenBank Accession No. NM_{—}005036) is expressed most in brown adipose tissue and liver, then kidney, heart and skeletal muscle. PPARγ (GenBank Accession No. NM_{—}005037) is mainly expressed in adipose tissue, and to a lesser extent in colon, the immune system and the retina. PPARβ is found in many tissues but the highest expression is in the gut, kidney and heart.

PPARs are ligand-dependent transcription factors: activation of target gene transcription depends on the binding of the ligand to the receptor. Some ligands are shared by the three isotypes, such as polyunsaturated fatty acids and probably oxidized fatty acids.

There are two varieties of diabetes. Type I is insulin-dependent diabetes mellitus (IDDM), for which insulin injection is required; it was formerly referred to as juvenile onset diabetes. In this type, insulin is not secreted by the pancreas and hence must be taken by injection. Type II, non-insulin-dependent diabetes mellitus (NIDDM) may be controlled by dietary restriction. It derives from insufficient pancreatic insulin secretion and tissue resistance to secreted insulin, which is complicated by subtle changes in the secretion of insulin by the beta cells. Despite their former classifications as juvenile or adult, either type can occur at any age; NIDDM, however, is the most common type, accounting for 90 percent of all diabetes.

While the exact causes of diabetes remain obscure, it is evident that NIDDM is linked to heredity and obesity. NIDDM is almost invariably accompanied by dyslipidemia, characterized by elevated triglycerides (TGs), VLDL-C and increased small dense LDL-C in combination with decreased levels of HDL-C and prolonged post-prandial hyperlipidemia. This form of dyslipidemia is highly atherogenic and thus represents a major risk factor for the development of premature atherosclerosis and coronary artery disease (CAD), which is the major cause of mortality in diabetic patients. A direct correlation between low HDL levels and incidence of CAD has been identified. In addition, this pathological lipid profile or “lipotoxicity” is suggested to contribute to β-cell failure and as a consequence impaired glucose stimulated insulin release.

Pharmacological, genetic and biochemical studies have unequivocally established that PPARα and PPARγ are key sensors and transcriptional modulators of lipid and glucose homeostasis, respectively. Accordingly, a selective “dual action drug” that selectively binds and activates PPARα and γ is hypothesized to mechanistically target the two major metabolic abnormalities observed in type II diabetic patients and thus therapeutically intervene with insulin resistance, CAD and possibly also impaired insulin secretion or β-cell failure.

Murakami et al. (1998) Diabetes 47: 1841-1847, discloses a thiazolidinedione derivative which activated both PPARα and PPARγ, and restored reduced lipid oxidation, when administered to obese rats. It was suggested that PPARα agonism has a protective effect against abnormal lipid metabolism in liver of obese rats. Agents modulating both PPARα and PPARγ are also disclosed in Shibata, T. et al. (1999) Eur. J. Pharmacol. 364: 211-219; and in WO 99/19313.

In EP 244097, relating to certain herbicidal pyrazolopyrimidine derivatives, the compound 2-[(phenylmethyl)thio]-5,7-bis(trifluoromethyl)-pyrazolo[1,5-a]pyrimidine-3-carbonitrile is disclosed.

The compound 5,7-bis(trifluoromethyl)-pyrazolo[1,5-a]pyrimidine-3-carbonitrile, was used by Chern et al. (1996) Chin. Pharm. J. 48: 37-52 for the synthetic preparation of pyrazolo-pyrimidinyl-oxadiazole derivatives as potential 5-HT3 antagonists. Siddiqi et al. (1996) Nucleosides Nucleotides 15(1-3), 693-717 showed that 2-(methylthio)-5,7-bis(trifluoromethyl)-pyrazolo[1,5-a]pyrimidine-3-carbonitrile and acts as an adenosine receptor ligand.

The compounds 3-cyano-α-phenylmethylene)-5,7-bis(trifluoromethyl)-pyrazolo[1,5-a]pyrimidine-2-acetonitrile (CAS RN: 338786-53-1), 3-cyano-5,7-bis(trifluoromethyl)-pyrazolo[1,5-a]pyrimidine-2-acetonitrile (CAS RN: 338786-45-1), and 3-cyano-α-[(dimethylamino)methylene]-5,7-bis(trifluoromethyl)-pyrazolo[1,5-a]pyrimidine-2-acetonitrile (CAS RN: 338786-57-5), are disclosed in a chemical library and are commercially available via Ambinter and Bionet Research.

### SUMMARY OF THE INVENTION

It has surprisingly been found that compounds of the general formula I, which are substituted derivatives of pyrazolo[1,5-a]pyrimidines, exhibits activity as modulators of peroxisome proliferator-activated receptors (PPAR) α and γ (PPAR modulators). The term “PPAR modulator” is intended to mean a PPAR ligand that is capable of acting as an activator (agonist), or alternatively as an inhibitor (antagonist), in PPAR mediated transcriptional responses.

Consequently, in a first aspect this invention provides a compound of the formula I

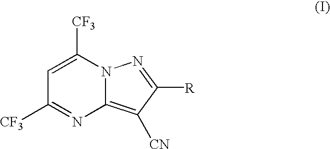

or a pharmaceutically acceptable salt or a prodrug form thereof, wherein R is

hydrogen,

C₁-6 alkylthio,

arylthio,

cyano-C₁₋₆ alkyl,

—C(CN)═CH—R¹ or

—CH(CN)—CH₂—R¹,

wherein R¹ is an aryl or heteroaryl group, optionally substituted in one or more positions with

halogen,

cyano,

nitro,

C₁₋₆ alkyl,

C₂₋₆ alkenyl,

C₁₋₆ alkoxy,

C₁₋₆ alkylthio,

C₁₋₆ alkylsulphonyl,

C₁₋₆ acyl,

hydroxy,

methylhydroxy,

carboxy,

formyl,

fluoromethyl,

difluoromethyl,

trifluoromethyl,

difluoromethoxy,

trifluoromethoxy,

difluoromethylthio,

trifluoromethylthio,

amino,

C₁₋₆ alkylamino,

di(C₁₋₆-alkyl)amino,

C₁₋₆ acylamino,

allyloxy,

aryl,

aryloxy,

benzyloxy, or

arylthio;

with the proviso that the said compound is not

5,7-bis(trifluoromethyl)-pyrazolo[1,5-a]pyrimidine-3-carbonitrile,

2-(methylthio)-5,7-bis(trifluoromethyl)-pyrazolo[1,5-a]pyrimidine-3-carbonitrile,

2-[(phenylmethyl)thio]-5,7-bis(trifluoromethyl)-pyrazolo[1,5-a]pyrimidine-3-carbonitrile,

3-cyano-α-(phenylmethylene)-5,7-bis(trifluoromethyl)-pyrazolo[1,5-a]pyrimidine-2-acetonitrile,

3-cyano-5,7-bis(trifluoromethyl)-pyrazolo[1,5-a]pyrimidine-2-acetonitrile,

3-cyano-α-[(dimethylamino)methylene]-5,7-bis(trifluoromethyl)-pyrazolo[1,5-a]pyrimidine-2-acetonitrile.

Preferred compounds of the formula I include those wherein R¹ is selected from the group consisting of, optionally substituted, phenyl, indenyl, naphthyl, thienyl, pyridinyl, quinoxalinyl, benzoylphenyl, thiazolyl, furyl, imidazolyl, oxazolyl, pyrazinyl, quinolinyl, indolyl, benzofuran, benzothiophenyl, pyrimidinyl, benzodioxolyl, provided that when R is —C(CN)═CH—R¹ and R¹ is phenyl, the phenyl is substituted.

R¹ is optionally and independently substituted in one or more positions with

halogen,

cyano,

nitro,

C₁₋₆ alkyl,

C₂₋₆ alkenyl,

C₁₋₆ alkoxy,

C₁₋₆ alkylthio,

C₁₋₆ alkylsulphonyl,

C₁₋₆ acyl,

hydroxy,

methylhydroxy,

carboxy,

formyl,

fluoromethyl,

difluoromethyl,

trifluoromethyl,

difluoromethoxy,

trifluoromethoxy,

difluoromethylthio,

trifluoromethylthio,

amino,

C₁₋₆ alkylamino,

di(C₁₋₆-alkyl)amino,

C₁₋₆ acylamino,

allyloxy,

aryl,

aryloxy,

benzyloxy,

arylthio, or

arylcarbonyl;

In particular, R¹ can be independently substituted in one or more positions with

chloro,

fluoro,

bromo,

iodo,

cyano,

nitro,

methyl,

ethyl,

isopropyl,

methoxy,

thiomethoxy

ethoxy,

methylsulfonyl,

acetyl,

methylhydroxy,

carboxy,

formyl,

trifluoromethyl,

trifluoromethoxy,

amino,

methylamino,

dimethylamino,

acetylamino,

phenyl,

benzyloxy,

phenoxy, or

benzoyl.

The following compounds are especially preferred:

3-cyano-α-(thenylidene)-5,7-bis(trifluoromethyl)-pyrazolo[1,5-a]pyrimidine-2-acetonitrile,

3-cyano-α-[(3-furyl)methylene]-5,7-bis(trifluoromethyl)-pyrazolo[1,5-a]pyrimidine-2-acetonitrile,

3-cyano-α-(furfurylidene)-5,7-bis(trifluoromethyl)-pyrazolo[1,5-a]pyrimidine-2-acetonitrile,

3-cyano-α-(4-methyl-5-propenyl-furfurylidene)-5,7-bis-trifluoromethyl-pyrazolo[1,5-a]pyrimidine-2-acetonitrile, or

3-cyano-α-(1-naphthylmethylene)-5,7-bis(trifluoromethyl)-pyrazolo[1,5-a]pyrimidine-2-acetonitrile.

In another aspect, this invention features a pharmaceutical formulation including at least one compound of the formula I as defined above, and a pharmaceutically acceptable diluent or carrier.

In further another aspect, this invention features a method for modulating (e.g., inhibiting) peroxisome proliferator-activated receptor α or γ activity. The method includes administering to a subject (e.g., mammal, human, or animal) in need thereof an effective amount of a compound of the formula I

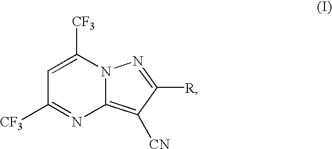

or a pharmaceutically acceptable salt thereof, wherein R is

hydrogen,

C₁₋₆ alkylthio,

arylalkylthio,

cyano-C₁₋₆ alkyl,

—C(CN)═CH—R¹ or

—CH(CN)—CH₂—R¹,

wherein R¹ is an aryl or heteroaryl group, optionally substituted in one or more positions with

halogen,

cyano,

nitro,

C₁₋₆ alkyl,

C₂₋₆ alkenyl,

C₁₋₆ alkoxy,

C₁₋₆ alkylthio,

C₁₋₆ alkylsulphonyl,

C₁₋₆ acyl,

hydroxy,

methylhydroxy,

carboxy,

formyl,

fluoromethyl,

difluoromethyl,

trifluoromethyl,

difluoromethoxy,

trifluoromethoxy,

difluoromethylthio,

trifluoromethylthio,

amino,

C₁₋₆ alkylamino,

di(C₁₋₆-alkyl)amino,

C₁₋₆ acylamino,

allyloxy,

aryl,

aryloxy,

benzyloxy, or

arylthio.

The exemplary compounds that can be used to practice the method of this invention include:

5,7-bis(trifluoromethyl)-pyrazolo[1,5-a]pyrimidine-3-carbonitrile,

2-(methylthio)-5,7-bis(trifluoromethyl)-pyrazolo[1,5-a]pyrimidine-3-carbonitrile,

2-[(phenylmethyl)thio]-5,7-bis(trifluoromethyl)-pyrazolo[1,5-a]pyrimidine-3-carbonitrile,

3-cyano-α-(phenylmethylene)-5,7-bis(trifluoromethyl)-pyrazolo[1,5-a]pyrimidine-2-acetonitrile,

3-cyano-5,7-bis(trifluoromethyl)-pyrazolo[1,5-a]pyrimidine-2-acetonitrile,

3-cyano-α-[(dimethylamino)methylene]-5,7-bis(trifluoromethyl)-pyrazolo[1,5-a]pyrimidine-2-acetonitrile,

3-cyano-α-(thenylidene)-5,7-bis(trifluoromethyl)-pyrazolo[1,5-a]pyrimidine-2-acetonitrile,

3-cyano-α-[(3-furyl)methylene]-5,7-bis(trifluoromethyl)-pyrazolo[1,5-a]pyrimidine-2-acetonitrile,

3-cyano-α-(furfurylidene)-5,7-bis(trifluoromethyl)-pyrazolo[1,5-a]pyrimidine-2-acetonitrile,

3-cyano-α-(4-methyl-5-propenyl-furfurylidene)-5,7-bis-trifluoromethyl-pyrazolo[1,5-a]pyrimidine-2-acetonitrile, or

3-cyano-α-(1-naphthylmethylene)-5,7-bis(trifluoromethyl)-pyrazolo[1,5-a]pyrimidine-2-acetonitrile.

Also within the scope of this invention is a method for making a compound of the formula I. The method includes taking any intermediate compound delineated herein, reacting it with any one or more reagents to form a compound of the formula I including any processes specifically delineated herein.

Other features and advantages of the invention will be apparent from the detailed description and the claims.

### DETAILED DESCRIPTION OF THE INVENTION

Definitions

The term “C₁₋₆ alkyl” denotes a straight or branched alkyl group having from 1 to 6 carbon atoms. Examples of said C₁₋₆ alkyl include methyl, ethyl, n-propyl, iso-propyl, n-butyl, iso-butyl, sec-butyl, t-butyl and straight- and branched-chain pentyl and hexyl. Derived expressions such as “C₁₋₆ alkoxy”, “C₁₋₆ alkylthio” and “C₁₋₆ alkylamino” are to be construed accordingly where an oxy group, thio group or an amino group, respectively, is bridging the C₁₋₆ alkyl group to the node at which that substituent is substituted.

The term “C₁₋₆ acyl” as used herein refers to the radical obtained by removal of hydroxyl from the carboxyl group in the corresponding carboxylic acid containing from 1 to 6 carbon atoms. Examples of said C₁₋₆ acyl include formyl, acetyl, butyryl, isobutyryl, and valeryl.

The term “halogen” shall mean fluorine, chlorine, bromine or iodine.

The term “aryl” denotes aromatic rings (monocyclic or bicyclic) having from 6 to 10 ring carbon atoms. Examples of said aryl include phenyl, indenyl and naphthyl.

The term “heteroaryl” denotes a mono- or bicyclic ring system (only one ring need to be aromatic, and substitution may be in any ring) having from 5 to 10 ring atoms, in which one or more of the ring atoms are other than carbon, such as nitrogen, oxygen and sulfur. Examples of said heteroaryl include pyrrole, thiazole, imidazole, thiophene, furan, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, quinazoline, indole, isoindole, isoindoline, indoline, benzothiophene, benzofuran, 2,3-dihydrobenzofuran, isobenzofuran, benzoxazole, 2,1,3-benzoxadiazole, benzothiazole, 2,1,3-benzothiadiazole, 2,1,3-benzoselenadiazole, benzimidazole, indazole, 2,3-dihydro-1,4-benzodioxine, indane, 1,3-benzodioxole, 3,4-dihydro-2H-1,4-benzoxazine, 1,5-naphthyridine, 1,8-naphthyridine.

The term “heteroalkyl chain” denotes a straight or branched, saturated or unsaturated, chain comprising from 1 to 4 carbon atoms and from 1 to 4 heteroatoms selected from the group consisting of O, N, and S. The heteroatom(s) may be placed at any position of the heteroalkyl group.

Depending on the process conditions the end products of the Formula I are obtained either in neutral or salt form. Both the free base and the salts of these end products are within the scope of the invention.

All diastereomeric forms possible (pure enantiomers, tautomers, racemic mixtures and unequal mixtures of two enantiomers) are within the scope of the invention. Such compounds can also occur as cis- or trans-, E- or Z-double bond isomer forms. All isomeric forms are contemplated.

The compounds of the formula I may be used as such or, where appropriate, as pharmacologically acceptable salts (acid or base addition salts) thereof.

The pharmacologically acceptable addition salts as mentioned above are meant to comprise the therapeutically active non-toxic acid and base addition salt forms which the compounds are able to form. Compounds which have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

Therapeutic or prophylactic treatment of mammals, including man, for conditions where modulation of either PPARα or PPARγ activity, or the combination of both PPARα and PPARγ activities, is of therapeutic benefit. Such conditions could be e.g. diabetes, diabetes mellitus type 2, insulin resistance, impaired glucose tolerance and/or in combinations with dyslipidemias, obesity, atherosclerosis, coronary artery disease, PCOS, gestational diabetes, inflammation.

The compounds according to the invention are particularly useful for the treatment of type II diabetes, in combination(s) with dyslipidemias, obesity, atherosclerosis and coronary artery disease. For this purpose the compounds according to the invention can be used alone or in combination(s) with sulfonylureas, metformin, alpha-glycosidase inhibitors, insulin or other anti-diabetic treatments/agents. Reference to treatment is intended to include prophylaxis as well as the alleviation of established symptoms.

For clinical use, the compounds of the invention are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. The formulations can be further prepared by known methods such as granulation, compression, microencapsulation, spray coating, etc.

The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

“An effective amount” refers to an amount of a compound which confers a therapeutic effect on the treated subject. The therapeutic effect may be objective (i.e., measurable by some test or marker) or subjective (i.e., subject gives an indication of or feels an effect). The dosage may, for example, range from about 0.1 mg to about 1000 mg per kilo of body weight, preferably from about 0.5 mg to about 500 mg per kilo of body weight, administered singly or multiply in doses. The typical daily dose of the active substance varies within a wide range and will depend on various factors such as for example the individual requirement of each patient and the route of administration.

The compounds according to the invention may also be administered as prodrugs that may be converted to the active ingredient in question after metabolic transformation in vivo. Conventional procedures for the selection and preparation of suitable prodrug derivatives are described, for example, in “Design of Prodrugs” ed. H. Bundgaard, Elsevier, 1985.

This invention relates to methods of making compounds of any of the formulae herein comprising reacting any one or more of the compounds of the formulae delineated herein, including any processes delineated herein. The compounds of the formula I above may be prepared by, or in analogy with, conventional methods, and especially according to or in analogy with the following methods.

Processes for Preparation

In a further aspect the invention provides a process for the preparation of a compound as defined above. The compounds according to the invention can be prepared by, or in analogy with, standard synthetic methods, and especially according to, or in analogy with, the following methods.

Method 1

Compound with formula (I) in scheme 1 is reacted with compounds with formula (II) at reflux temperature in presence of piperidine using EtOH as solvent to give compounds with formula (III). Further reaction with 1,1,1,5,5,5-hexafluoro-pentane-2,4-dione at reflux temperature, using glacial acetic acid as solvent, affords the target compound (IV).

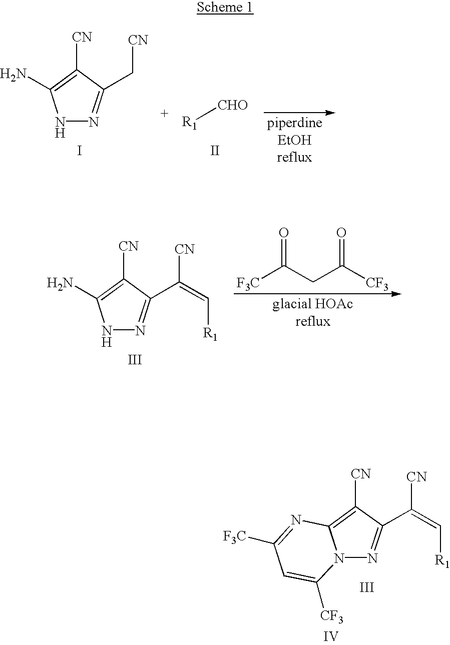

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLES

Synthetic Methods

The structures of the prepared compounds were confirmed by standard spectroscopical methods. The NMR data was obtained on a Jeol JNM-EX 270 or a Bruker DRX 500 spectrometer. Electrospray MS data was obtained on a Jeol SX102A or a Quattro I or a LCT (KA011) mass spectrometer.

### Example 1

3-Cyano-α-(thenylidene)-5,7-bis(trifluoromethyl)-pyrazolo[5-a]pyrimidine-2-acetonitrile

Step 1: 5-Amino-3-[1-cyano-2-(2-thienyl)ethenyl]-1H-pyrazole-4-carbonitrile

To a stirred solution of 5-amino-3-(cyanomethyl)-1H-pyrazole-4-carbonitrile (1 eq) in EtOH, piperidine (0.5 eq) was added followed by 2-thiophenealdehyde. The solution was heated to reflux. After 4 hours the reaction mixture was allowed to cool and a precipitate was formed giving 5-amino-3-[1-cyano-2-(2-thienyl)ethenyl]-1H-pyrazole-4-carbonitrile in a 55% yield. ¹HNMR (DMSO-d₆) δ 14.4 (br, 1H), 8.04 (s, 1H), 7.95 (d, 1H), 7.72 (d, 1H), 7.25 (dd, 1H), 6.69 (br, 2H); MS m/z 242 (M+1)

Step 2: 3-Cyano-α-(thenylidene)-5,7-bis(trifluoromethyl)-pyrazolo[1,5-a]pyrimidine-2-acetonitrile

5-Amino-3-[1-cyano-2-(2-thienyl)ethenyl]-1H-pyrazole-4-carbonitrile was reacted with hexafluoropentadione (1.5 eq) in glacial HOAc under reflux temperature for 2 hours. A precipitate was formed that was filtered off giving the desired product in 92% yield. ¹HNMR (DMSO-d₆) δ 8.63 (s, 1H), 8.55 (s, 1H), 8.20 (d, 1H), 8.07 (d, 1H), 7.39 (dd, 1H); MS m/z 413 (M+1)

### Example 2

3-Cyano-α-[(3-furyl)methylene]-5,7-bis(trifluoromethyl)-pyrazolo[1,5-a]pyrimidine-2-acetonitrile

Step 1: 5-Amino-3-[1-cyano-2-(3-furyl)ethenyl]-1H-pyrazole-4-carbonitrile

Use of 3-furylaldehyde according to method 1 afforded 0.14 g (60%) of the title compound. ¹HNMR (DMSO-d₆) δ 12.42 (br, 1H), 8.03-8.02 (m, 1H), 7.64 (s, 1H), 7.24 (d, 1H), 6.78-6.76 (m, 1H), 6.69 (br, 2H)

Step 2: 3-Cyano-α-[(3-furyl)methylene]-5,7-bis(trifluoromethyl)-pyrazolo[1,5-a]pyrimidine-2-acetonitrile

The crude product was purified by column chromatography on silica gel using n-hexane: EtOAc 8:2 giving 0.08 g (33%) of the title compound. ¹HNMR (DMSO-d₆) δ 8.68 (s, 1H), 8.57 (s, 1H), 8.37 (s, 1H), 8.01-8.00 (m, 1H), 7.32-7.31 (m, 1H)

### Example 3

3-Cyano-α-(furfurylidene)-5,7-bis(trifluoromethyl)-pyrazolo[1,5-a]pyrimidine-2-acetonitrile

Step 1: 5-Amino-3-[1-cyano-2-(2-furyl)ethenyl]-1H-pyrazole-4-carbonitrile

Use of 2-furylaldehyde according to method 1 afforded 0.15 g (67%) of the title compound, 60% pure according to HPLC analysis, used in next step without further purification.

Step 2: 3-Cyano-α-(furfurylidene)-5,7-bis(trifluoromethyl)-pyrazolo[1,5-a]pyrimidine-2-acetonitrile

The crude product was purified by column chromatografy on silica gel using n-hexane: EtOAc 8:2 giving 0.02 g (7%) of the title compound. ¹HNMR (DMSO-d₆) δ 8.56 (s, 1H), 8.25-8.24 (m, 1H), 8.22 (s, 1H), 7.58 (d, 1H), 6.92-6.90 (m 1H)

### Example 4

3-cyano-α-(1-naphthylmethylene)-5,7-bis(trifluoromethyl)-pyrazolo[1,5-a]pyrimidine-2-acetonitrile

Step 1: 5-Amino-3-[1-cyano-2-(1-naphtyl)ethenyl]-1H-pyrazole-4-carbonitrile

Use of 1-naphtylaldehyde according to method 1 afforded 0.1 g (55%) of the title compound. ¹HNMR (DMSO-d₆) δ 8.60 (s, 1H), 8.12-8.03 (m, 4H), 7.67-7.60 (m, 4H), 6.79 (s, 2H)

Step 2: 3-cyano-α-(1-naphthylmethylene)-5,7-bis(trifluoromethyl)-pyrazolo[1,5-a]pyrimidine-2-acetonitrile

Purification by column chromatography on silica gel using n-hexane: EtOAc 8:2 gave 0.02 g (13%) of the title compound.

¹HNMR (DMSO-d₆) δ 9.21 (s, 1H), 8.61 (s, 1H), 8.32 (d, 1H), 8.24-8.19 (m, 2H), 8.12 (d, 1H), 7.78-7.75 (m, 2H), 7.71-7.68 (m, 1H); MS m/z 458 (M+1)

### Example 5

3-cyano-α-(4-methyl-5-propenyl-furfurylidene)-5,7-bis-trifluoromethyl-pyrazolo[1,5-a]pyrimidine-2-acetonitrile

Step 1: 5-Amino-3-(2-benzofuran-2-yl-1-cyano-vinyl)-1H-pyrazole-4-carbonitrile

Use of 1-benzofuran-2-carbaldehyde according to method 1 afforded 0.1 g (48%) of the title compound. ¹H NMR (acetic acid-d₄) δ 8.04 (s, 1H), 7.68-7.51 (m, 4H), 7.34-7.28 (m, 1H).

Step 2: 3-cyano-α-(4-methyl-5-propenyl-furfurylidene)-5,7-bis-trifluoromethyl-pyrazolo[1,5-a]pyrimidine-2-acetonitrile

Filtration of the precipitate afforded 0.06 g (15%) of the title compound. ¹H NMR (DMSO-d₆) δ 8.58 (s, 1H), 8.36 (s, 1H), 7.99 (s, 1H), 7.88 (d, J=7.9 Hz, 1H), 7.75 (d, J=8.5 Hz, 1H), 7.60-7.56 (m, 1H), 7.42-7.40 (m, 1H); MS m/z 448 (M+H)⁺.

### Comparative Example

2-(Methylthio)-5,7-bis(trifluoromethyl)pyrazolo[1,5-a]pyrimidine-3-carbonitrile (PNU-242580)

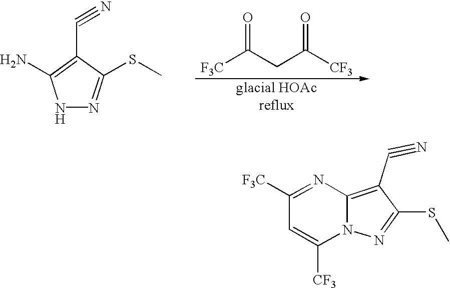

5-amino-3-(methylthio)-1H-pyrazole-4-carbonitrile (0.5 g, 3.24 mmol) was refluxed at 120° C. in glacial HOAc for 4 hours. The reaction mixture was allowed to cool. The obtained precipitate was filtered off giving 0.75 g (75%) of the title compound. ¹HNMR (CDCl₃) δ 7.55 (s, 2H), 2.77 (s, 3H); ¹³CNMR (CDCl₃) δ 163.7, 150.8, 149.9, 149.6, 149.3, 149.0, 135.9, 135.7, 121.6, 120.6, 119.4, 118.4, 117.2, 115.1, 110.1, 103.4, 85.3, 13.6.

Biological Methods

The modulation activity of a candidate compound can be determined in a number of testing protocols (e.g., in vivo, in vitro), screens and assays, including those delineated herein, known in the art.

(I) Cell-Based Reporter Assays

The effect of compounds according to the invention on activation of PPARα and PPARγ were determined. Reporter gene assays were performed essentially as described in Bertilsson et al., 1998 (Proc. Natl. Acad. Sci. U.S.A. 95:12208-12213), by transient co-transfections of CaCo2/TC cells with a GAL-4-LBD (Ligand Binding Domain) fusion constructs, containing the nucleotide sequence corresponding to human PPARαLBD (i.e. amino acid residues 167-468) or human PPARγLBD (i.e. amino acid residues 204-477), together with a 4×UAS-luciferase reporter gene construct, using the FuGENE-6 transfection reagent (Roche) according to the manufacturers recommendations. After 24 hours, the cells were treated with trypsin, transferred to 96-well microplates and allowed to settle. Induction was performed for 24 hours by applying different concentrations of compounds diluted in DMSO or DMSO alone (vehicle). Subsequently, the cells were lysed and luciferase activity measured, according to standard procedures. Experiments were performed in quadruplicate on at least three occasions.

The compounds of formula I exhibit EC₅₀ values on PPARα and PPARγ in the range of 1-35 μM and 0.3-50 μM, respectively.

(II) Ligand Binding Assays

Crude extracts were prepared from E. coli (BL21(DE3)pLysS, Novagen) producing GST-PPARαLBD or GST-PPARγLBD fusion proteins by freeze thawing in buffer containing 50 mM Tris-HCl pH 7.9, 250 mM KCl, 10% glycerol, 1% Triton X-100, 10 mM DTT, 1 mM PMSF, 10 μg/mL DNase and 10 mM MgCl. Competitive ligand binding assays were performed on immobilized GST-PPARαLBD or GST-PPARγLBD fusion proteins from crude extracts incubated with glutathione-Sepharose 4B (Amersham Pharmacia Biotech). Following immobilization, the slurry was washed three times in binding buffer containing 50 mM Tris-HCL, pH 7.9, 50 mM KCl, 0.1% Triton-X100, 10 mM DTT, 2 mM EDTA, dispensed in 96-well filter plates (MHVB N45, Millipore) and incubated with a fixed amount tritiated ligand and different concentrations of cold competing ligands. Equilibrium binding was reached after incubation for 2 hours at room temperature on a plate shaker. The plates were then washed 3 times in binding buffer, dried overnight at room temperature followed by scintillation counting after the addition of 25 μl of scintillant (Optiscint Hisafe, Wallac) per well. Each experiment was performed in duplicate and repeated independently at least three times. ³H-BRL49653 (ART-605; American Radiolabeled Chemicals, USA) was used as the tracer in PPARγ competitive ligand binding experiments at a concentration of 30 nM (10). ³H-GW2331 (70 nM) was synthesized at Pharmacia Corporation and used as the tracer in PPARα competitive ligand binding experiments (Kliewer, S. A. et al. (1997) Proc. Natl. Acad. Sci. U.S.A. 94: 4318-4323).

The compounds of formula I exhibit Kᵢ values and EC₅₀ in the range of 1-100 μM and 0.1-100 μM, respectively, on both PPARα and PPARγ.

What is claimed is:

1. A compound of the formula I 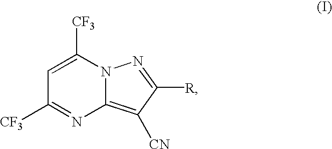
or a pharmaceutically acceptable salt thereof, wherein R is
hydrogen,
C₁₋₆ alkylthio,
arylalkylthio,
cyano-C₁₋₆ alkyl,
—C(CN)═CH—R¹ or
—CH(CN)—CH₂—R¹,
wherein R¹ is an aryl or heteroaryl group, optionally substituted in one or more positions with
halogen,
cyano,
nitro,
C₁₋₆ alkyl,
C₂₋₆ alkenyl,
C₁₋₆ alkoxy,
C₁₋₆ alkylthio,
C₁₋₆ alkylsulphonyl,
C₁₋₆ acyl,
hydroxy,
methylhydroxy,
carboxy,
formyl,
fluoromethyl,
difluoromethyl,
trifluoromethyl,
difluoromethoxy,
trifluoromethoxy,
difluoromethylthio,
trifluoromethylthio,
amino,
C₁₋₆ alkylamino,
di(C₁₋₆-alkyl)amino,
C₁₋₆ acylamino,
allyloxy,
aryl,
aryloxy,
benzyloxy, or
arylthio;
with the proviso that the said compound is not
5,7-bis(trifluoromethyl)-pyrazolo[1,5-a]pyrimidine-3-carbonitrile,
2-(methylthio)-5,7-bis(trifluoromethyl)-pyrazolo[1,5-a]pyrimidine-3-carbonitrile,
2-[(phenylmethyl)thio]-5,7-bis(trifluoromethyl)-pyrazolo[1,5-a]pyrimidine-3-carbonitrile,
3-cyano-α-(phenylmethylene)-5,7-bis(trifluoromethyl)-pyrazolo[1,5-a]pyrimidine-2-acetonitrile,
3-cyano-5,7-bis(trifluoromethyl)-pyrazolo[1,5-a]pyrimidine-2-acetonitrile, or
3-cyano-α-[(dimethylamino)methylene]-5,7-bis(trifluoromethyl)-pyrazolo[1,5-a]pyrimidine-2-acetonitrile.

2. The compound according to claim 1, wherein R is
—C(CN)═CH—R¹ or
—CH(CN)—CH₂—R¹
wherein R¹ is selected from the group consisting of, optionally substituted, phenyl, indenyl, naphthyl, thienyl, pyridinyl, quinoxalinyl, benzoylphenyl, thiazolyl, furyl, imidazolyl, oxazolyl, pyrazinyl, quinolinyl, indolyl, benzofuran, benzothiophenyl, pyrimidinyl, benzodioxolyl;
and R¹ is optionally and independently substituted in one or more positions with
halogen,
cyano,
nitro,
C₁₋₆ alkyl,
C₂₋₆ alkenyl,
C₁₋₆ alkoxy,
C₁₋₆ alkylthio,
C₁₋₆ alkylsulphonyl,
C₁₋₆ acyl,
hydroxy,
methylhydroxy,
carboxy,
formyl,
fluoromethyl,
difluoromethyl,
trifluoromethyl,
difluoromethoxy,
trifluoromethoxy,
difluoromethylthio,
trifluoromethylthio,
amino,
C₁₋₆ alkylamino,
di(C₁₋₆-alkyl)amino,
C₁₋₆ acylamino,
allyloxy,
aryl,
aryloxy,
benzyloxy,
arylthio, or
arylcarbonyl;
with the proviso that when R is —C(CN)═CH—R¹, then R¹ is not unsubstituted phenyl.

3. The compound according to claim 1 wherein R¹ can be independently substituted in one or more positions with
chloro,
fluoro,
bromo,
iodo,
cyano,
nitro,
methyl,
ethyl,
isopropyl,
methoxy,
thiomethoxy
ethoxy,
methylsulfonyl,
acetyl,
methylhydroxy,
carboxy,
formyl,
trifluoromethyl,
trifluoromethoxy,
amino,
methylamino,
dimethylamino,
acetylamino,
phenyl,
benzyloxy,
phenoxy, or
benzoyl.

4. The compound according to claim 1 wherein R¹ is
aryl-C₁₋₆-alkyl,
furyl-C₁₋₆-alkyl, or
thienyl-C₁₋₆-alkyl.

5. The compound according to claim 1 which is the compound
3-cyano-α-(thenylidene)-5,7-bis(trifluoromethyl)-pyrazolo[1,5-a]pyrimidine-2-acetonitrile,
3-cyano-α-[(3-furyl)methylene]-5,7-bis(trifluoromethyl)-pyrazolo[1,5-a]pyrimidine-2-acetonitrile,
3-cyano-α-(furfurylidene)-5,7-bis(trifluoromethyl)-pyrazolo[1,5-a]pyrimidine-2-acetonitrile,
3-cyano-α-(4-methyl-5-propenyl-furfurylidene)-5,7-bis-trifluoromethyl-pyrazolo[1,5-a]pyrimidine-2-acetonitrile, or
3-cyano-α-(1-naphthylmethylene)-5,7-bis(trifluoromethyl)-pyrazolo[1,5-a]pyrimidine-2-acetonitrile.

6. A process for the preparation of a compound according to claim 1, wherein R is —C(CN)═CH—R¹, comprising a first step wherein 5-amino-3-(cyanomethyl)-1H-pyrazole-4-carbonitrile is reacted with an aldehyde and a second step comprising the reaction of hexafluoropentadione with the reaction product of the first step, wherein the aldehyde is of formula R¹—CHO and R¹ is an aryl or heteroaryl group, optionally substituted in one or more positions with
halogen,
cyano,
nitro,
C₁₋₆ alkyl,
C₂₋₆ alkenyl,
C₁₋₆ alkoxy,
C₁₋₆ alkylthio,
C₁₋₆ alkylsulphonyl,
C₁₋₆ acyl,
hydroxy,
methylhydroxy,
carboxy,
formyl,
fluoromethyl,
difluoromethyl,
trifluoromethyl,
difluoromethoxy,
trifluoromethoxy,
difluoromethylthio,
trifluoromethylthio,
amino,
C₁₋₆ alkylamino,
di(C₁₋₆-alkyl)amino,
C₁₋₆ acylamino,
allyloxy,
aryl,
aryloxy,
benzyloxy, or
arylthio.

7. The process according to claim 6 comprising a first step wherein 5-amino-3-(cyanomethyl)-1H-pyrazole-4-carbonitrile is reacted with an aldehyde at a reflux temperature in presence of piperidine using EtOH as the solvent and a second step comprising the reaction of 1,1,1,5,5,5-hexafluoro-pentane-2,4-dione with the reaction product of the first step at a reflux temperature using glacial acetic acid as the solvent, wherein the aldehyde is of formula R¹—CHO and R¹ is
aryl-C₁₋₆-alkyl,
furyl-C₁₋₆-alkyl, or
thienyl-C₁₋₆-alkyl.

8. The process according to claim 6 comprising a first step wherein 5-amino-3-(cyanomethyl)-1H-pyrazole-4-carbonitrile is reacted with an aldehyde and a second step comprising the reaction of 1,1,1,5,5,5-hexafluoro-pentane-2,4-dione with the reaction product of the first step, wherein the aldehyde is of formula R¹—CHO and R¹ is
aryl-C₁₋₆-alkyl,
furyl-C₁₋₆-alkyl, or
thienyl-C₁₋₆-alkyl.

9. A pharmaceutical formulation comprising a compound according to claim 1 as an active ingredient in combination with a pharmaceutically acceptable diluent or carrier.

10. A method for modulating peroxisome proliferator-activated receptor α or γ activity, comprising administering to a subject in need thereof an effective amount of a compound of the formula I 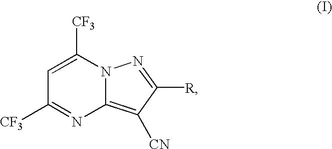
or a pharmaceutically acceptable salt thereof, wherein R is
hydrogen,
C₁₋₆ alkylthio,
arylalkylthio,
cyano-C₁₋₆alkyl,
—C(CN)═CH—R¹ or
—CH(CN)—CH₂—R¹,
wherein R¹ is an aryl or heteroaryl group, optionally substituted in one or more positions with
halogen,
cyano,
nitro,
C₁₋₆ alkyl,
C₂₋₆ alkenyl,
C₁₋₆ alkoxy,
C₁₋₆ alkylthio,
C₁₋₆ alkylsulphonyl,
C₁₋₆ acyl,
hydroxy,
methylhydroxy,
carboxy,
formyl,
fluoromethyl,
difluoromethyl,
trifluoromethyl,
difluoromethoxy,
trifluoromethoxy,
difluoromethylthio,
trifluoromethylthio,
amino,
C₁₋₆ alkylamino,
di(C₁₋₆-alkyl)amino,
C₁₋₆ acylamino,
allyloxy,
aryl,
aryloxy,
benzyloxy, or
arylthio.

11. The method according to claim 10, wherein the said compound is
5,7-bis(trifluoromethyl)-pyrazolo[1,5-a]pyrimidine-3-carbonitrile,
2-(methylthio)-5,7-bis(trifluoromethyl)-pyrazolo[1,5-a]pyrimidine-3-carbonitrile,
2-[(phenylmethyl)thio]-5,7-bis(trifluoromethyl)-pyrazolo[1,5-a]pyrimidine-3-carbonitrile,
3-cyano-α-(phenylmethylene)-5,7-bis(trifluoromethyl)-pyrazolo[1,5-a]pyrimidine-2-acetonitrile,
3-cyano-5,7-bis(trifluoromethyl)-pyrazolo[1,5-a]pyrimidine-2-acetonitrile,
3-cyano-α-[(dimethylamino)methylene]-5,7-bis(trifluoromethyl)-pyrazolo[1,5-a]pyrimidine-2-acetonitrile,
3-cyano-α-(thenylidene)-5,7-bis(trifluoromethyl)-pyrazolo[1,5-a]pyrimidine-2-acetonitrile,
3-cyano-α-[(3-furyl)methylene]-5,7-bis(trifluoromethyl)-pyrazolo[1,5-a]pyrimidine-2-acetonitrile,
3-cyano-α-(furfurylidene)-5,7-bis(trifluoromethyl)-pyrazolo[1,5-a]pyrimidine-2-acetonitrile,
3-cyano-α-(4-methyl-5-propenyl-furfurylidene)-5,7-bis-trifluoromethyl-pyrazolo[1,5-a]pyrimidine-2-acetonitrile, or
3-cyano-α-(1-naphthylmethylene)-5,7-bis(trifluoromethyl)-pyrazolo[1,5-a]pyrimidine-2-acetonitrile.

12. A method for treatment or prevention of diabetes and/or dyslipidemia comprising administering to a mammal, including a human, in need of such treatment an effective amount of a compound of the formula I 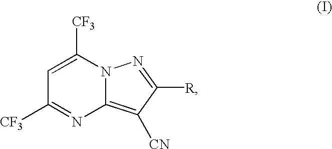
or a pharmaceutically acceptable salt thereof, wherein R is
hydrogen,
C₁₋₆ alkylthio,
arylalkylthio,
cyano-C₁₋₆ alkyl,
—C(CN)═CH—R¹ or
—CH(CN)—CH₂—R¹,
wherein R¹ is an aryl or heteroaryl group, optionally substituted in one or more positions with
halogen,
cyano,
nitro,
C₁₋₆ alkyl,
C₂₋₆ alkenyl,
C₁₋₆ alkoxy,
C₁₋₆ alkylthio,
C₁₋₆ alkylsulphonyl,
C₁₋₆ acyl,
hydroxy,
methylhydroxy,
carboxy,
formyl,
fluoromethyl,
difluoromethyl,
trifluoromethyl,
difluoromethoxy,
trifluoromethoxy,
difluoromethylthio,
trifluoromethylthio,
amino,
C₁₋₆ alkylamino,
di(C₁₋₆-alkyl)amino,
C₁₋₆ acylamino,
allyloxy,
aryl,
aryloxy,
benzyloxy, or
arylthio.
